(19) United States
(12) Patent Application Publication
(10) Pub. No.: US 2007/0088390 A1
(43) Pub. Date: Apr. 19, 2007

(54) Surgical fasteners and devices for surgical fastening

(75) Inventors: Adrian Paz, Petach Tikva (IL); Dan Rotenberg, Haifa (IL); Eitan Hod, Ya'akov (IL); Anatoli Konik, Haifa (IL); Mark Shahar, Tel-Aviv (IL); Roni Shabat, Tachton (IL)

Correspondence Address: NATH & ASSOCIATES, 112 South West Street, Alexandria, VA, 22314, US

(73) Assignee: ENDOGUN MEDICAL SYSTEMS LTD., Kiriat Shmona (IL)

(21) Appl. No.: 10/563,974

(22) PCT Filed: Jul. 11, 2004
(86) PCT No.: PCT/IL04/00624

§ 371 Date: Aug. 7, 2006

Related U.S. Application Data
(60) Provisional application No. 60/486,264, filed on Jul. 11, 2003.

(51) Int. Cl. A61B 17/04

(52) U.S. Cl. 606/232

(57) ABSTRACT

A surgical fastening device for pinning a surgical filament to a body tissue. The device includes a grasping handle and a slender shaft extending from the grasping handle. A compartment contains one or more surgical fasteners. An ejecting mechanism is used to eject a surgical fastener from a compartment containing one or more surgical fasteners. The device also includes a filament dispensing system that dispenses surgical filament along the shaft so that a fastener grasps the filament when being ejected from the shaft. The invention also provides surgical fasteners and surgical filaments for use in the device.

### FIELD OF THE INVENTION

This invention relates to surgical fasteners and to surgical fastening devices.

### BACKGROUND OF THE INVENTION

Surgical fasteners are used instead of surgical suturing, which is often both time consuming and inconvenient, in order to join two tissue locations. A surgeon can often use a stapling apparatus to implant a fastener into a body tissue and thus accomplish in a few seconds, what would take a much longer time to suture. A surgical fastener is used, for example in inguinal hernia surgery to fasten polypropylene mesh to the abdominal wall in order to reinforce the abdominal wall.

Conventional surgical fasteners have been in the form of ordinary metal staples, which are bent by the delivery apparatus to join together body tissues. These staples comprise a pair of legs or prongs joined together at one end by a crown that may be straight or arcuate.

At present, there are a variety of surgical fasteners and fastening devices available for endoscopic or open procedures, to attach tissues together, or to attach a mesh patch to a tissue. One such surgical fastener is a surgical stapler, or clip applicator. In this stapler, a plurality or stack of unformed staples are contained within a cartridge and are sequentially advanced or fed within the instrument by a spring mechanism. A secondary feeding mechanism is employed to separate the distal most staple from the stack, and to feed the distal most stapler into the staple closing mechanism. Such mechanisms are found in U.S. Pat. Nos. 5,470,010, and 5,582,616.

In some applications, the body tissue is accessible from two opposite direction so that an anvil may be used to deform the legs of a staple after having passed through the body tissue. In applications where access to the tissue is from only one direction, an anvil may be used to deform the crown of a conventional staple so that the legs project towards each other in the body tissue so as to hold the staple in the tissue.

Another stapler mechanism, used mostly for mesh attachment to tissue does not use an anvil. Instead, a fastener comprising a helical wire is screwed or rotated into a tissue, in order to join tissues to affix a polypropylene or similar material mesh or other patch to the tissue together. Instruments and fasteners of this type are found in U.S. Pat. No. 5,582,616, U.S. Pat. No. 5,810,882, and U.S. Pat. No. 5,830,221. Another type of fastener that does not need an anvil applies fasteners made from a shape memory alloy such as Nitinol^{T}. These fasteners are mainly used to fasten prosthetic material or artificial mesh to tissue.

In the above instruments, a mechanism is used that is located in a slender shaft of the instrument to push the stack of staples or anchors to the distal end of the shaft as the staples are ejected from the distal end. This mechanism prevents the shaft diameter from being reduced below a minimal diameter required to contain the mechanism. The minimum shaft diameter attainable with these instruments can limit the efficiency of some laparoscopic and minimally invasive procedures.

### SUMMARY OF THE INVENTION

In its first aspect the invention provides a surgical fastening device. As used herein, the phrase “surgical fastening device” refers to any surgical device for inserting a surgical fastener into a body tissue and includes surgical staplers, and surgical joiners. As used herein, the phrase “surgical fastener” refers to any device configured to be inserted and anchored into a body tissue and includes, for example, surgical staples, surgical pins, surgical anchors, surgical arrows and other types of fasteners used to join two tissues together, or to attach a synthetic device to a body tissue.

The surgical fastener of the invention is provided with an amount of a surgical filament. The fastener is configured to be inserted into a body cavity and to eject a surgical fastener so as to pin an end of a piece of the surgical filament to a body tissue in a first location in the cavity. The fastening device may then be moved to a second location in the cavity and a second fastener ejected from the device so as to pin another point on the filament to a body tissue at the second location in the cavity. The first and second locations of body tissue are thus connected to each other by a segment of the filament. The device is further configured to cut the filament so as to release the piece of the filament pinned at each of its ends to the first and second locations.

As used herein the term “surgical filament” refers to a filament having any cross sectional shape and made from a biocompatible material. The filament may have a rectangular cross section, such as a ribbon, band or strip, or may have a circular cross section, such as a cord, thread or wire. The filament may also be a hollow cylinder. The filament may be complete or may have perforations in it. The filament may also be a mesh or a net. The filament may be biodegradable or may be non-biodegradable.

The device of the invention may be used to attach one or more pieces of surgical filament into a body cavity in order to position an organ in the cavity, or to form a lattice of filaments to support a body tissue.

In its second aspect, the invention provides surgical fasteners for use in the surgical fastener of the invention.

In its third aspect, the invention provides surgical filaments for use in the surgical fastener of the invention.

Laparoscopic repair of inguinal hernia, for example, may be performed with the fastening device of the present invention using only one port. Abdominal wall defects can be closed using filaments attached to the tissue using the proper tension, so as to prevent recurrence. The approach may be properitoneal, using a dissection balloon and creating a lattice-like structure from pieces of filament that closes the defect of the abdominal wall from inside using pieces of filament and attaching them to the tissue. Alternatively, hernia repair may be performed endoscopically through a small skin incision. The defect is closed from the exterior side and then a space is created with a balloon inserted above the posterior wall of the inguinal canal. The weakness of the posterior wall of the inguinal canal is closed with the filaments attached to the tissue. This procedure reproduces endoscopically the open Lichtenstein mesh hernia repair operation, and may be performed under local anesthesia.

Another type of surgery that can benefit from the device of the invention is urinary stress incontinence. Presently, open abdominal operation for treating urinary stress incontinence is performed through a large incision in the lower abdomen; the vaginal wall is sutured to the Cooper's ligaments situated on the pubic bone creating a hammock and support for the urethra and preventing stress incontinence. Alternatively, a vaginal incision and two small abdominal incisions are performed for inserting an elastic strip beneath the urethra and fasten it to the pelvic bone or other hard tissue such as the rectus sheath fascia, in order to support the urethra and stop uncontrolled urine.

In contrast to this, with the device of the invention, through one small vaginal incision, a sling or mesh strip can be inserted beneath the urethra, fixed with fasteners to the rectus sheath fascia tissue or bone without any additional incisions in the abdominal wall. Alternatively, the operation can be performed through a small abdominal incision. The extraperitoneal space before the urinary bladder is developed using a dissection balloon. The anterior vaginal wall is attached to the Cooper ligaments or to pubic bone. A filament is attached to the vaginal wall using one or more fasteners. Then the filament is tensioned and attached with one or more such fasteners to the cooper ligament or to the pubic bone. One or more such filaments are used on each side, reproducing the open Burch intervention, laparoscopically using only one or two ports.

The device of the invention may also be used, for minimal invasive repair of pelvic organ prolapse. Such interventions are performed presently through a large abdominal or vaginal incision.

Laparoscopic repair or pelvic organ prolapse may be performed with the present device using only one or two ports. The pelvic floor defects can be closed using filaments attached to the tissue using the proper tension, thus preventing recurrence.

Another application of the present invention is in the repair of gastroesophageal reflux. The posterior wall of the stomach is sutured to the anterior wall on the medial side of the esophagus creating a valve like structure and preventing gastroesophageal reflux (Nissen fundoplication). The operation with the device of the present invention can be performed by one operator using one or two ports. The instrument is introduced through the lesser omentum and the filament is attached to the posterior wall of the stomach that is then pulled toward the anterior wall of the stomach medial to the esophagus. Then the filament is properly tensioned and attached to the anterior wall reproducing Nissen fundoplication.

The present invention may also be used for performing laparoscopic anastomoses of various tubular organs such as intestines or blood vessels, or for closing defects in such structures. The present invention may also be used to close defects in tubular organs such as intestines, stomach, or urinary bladder by an endoscopic route (from inside). Such interventions may be performed using local anesthesia during gastroscopy, colonoscopy or cystoscopy. Endoscopic excision of large tumors in these organs may create defects which may be closed using filaments attached to one side of the defect with attachment means, then the filament and the tissue affixed to it is approximated to the other tissue (the other lip of the defect) and the filament is attached to this tissue under proper tension.

Another intervention that may be performed with this instrument is endoscopic repair of ureteropelvic obstruction that is the most frequent inborn urinary tract anomaly. Currently, the gold standard for this operation is open pyeloplasty, that is performed under general anesthesia. Endoscopic repair of this anomaly can be performed by an antegrade or retrograde route, under local anesthesia. However, the results are inferior to the open repair since the defect created by incising the stricture is left open and can lead to restenosis.

Another endoscopic operation for the repair of ureteropelvic junction obstruction, endopyeloplasty, involves endoscopically incising the ureteropelvic junction longitudinally and closing the endoscopically in a transverse manner. With the prior art instruments, a suture is passed through the lips of the defect, the instrument is then removed and a knot is made extracorporeally and pushed down and then another instrument is introduced for cutting the suture thread. This sequence of maneuvers is performed for each suture 4 to 6 times. This procedure necessitates forming a large orifice in the flank and kidney for introducing the suturing instrument.

The fastening device of the invention may also be used in vaginal repair of stress incontinence. With the device of the invention, this procedure may be carried out under local anesthesia with reduced risk of injury to blood vessels, the urethra, urinary bladder bowels or nerves, which is known to occur during trans-abdominal or trans-vaginal surgery.

The fasteners of the present invention can be introduced under local anaesthesia through a small incision in the renal pelvis through the flank with ultrasound or fluoroscopic guidance. The strictured area can be incised longitudinally and the defect created may be closed transversally by attaching a biodegradable filament made, for example, from Vycril to one lip of the defect with anchors tensioning it, attaching the filament to the other lip, and closing the defect from inside. Since, the instrument may be as slender as 2 to 3 mm, the intervention may be performed expeditiously through a 5 mm orifice in the flank under local anesthesia.

Thus, in its first aspect, the invention provides a surgical fastening device for pinning a surgical filament to a body tissue, comprising:
(a) a grasping handle;
(b) a slender shaft extending from the grasping handle,
(c) a compartment configured to contain one or more surgical fasteners;
(d) an activatable ejecting mechanism ejecting a surgical fastener from the compartment; and
(e) a filament dispensing system configured to dispense surgical filament along the shaft so that a fastener grasps the filament when being ejected from the shaft.

In its second aspect, the invention provides a surgical fastener for use in the surgical fastening device of the invention.

In its third aspect, the invention provides a surgical filament for use in the surgical fastening device of the invention.

In its forth aspect, the invention provides a method for pinning a surgical filament to a first location of body tissue in a body cavity comprising introducing into the body cavity a surgical fastening device of the invention and ejecting a first surgical fastener from the shaft so as to pin a surgical filament to the first location.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a surgical fastener in accordance with one embodiment of the invention;

FIG. 2 shows a surgical fastener in accordance with a second embodiment of the invention;

FIG. 3 shows a surgical fastener in accordance with a third embodiment of the invention;

FIG. 4 shows a surgical fastener in accordance with a fourth embodiment of the invention;

FIG. 5 shows a surgical fastener in accordance with a fifth embodiment of the invention;

FIG. 6 shows a surgical fastening device in accordance with one embodiment of the invention;

FIG. 7 shows the distal end of the barrel of the device of FIG. 6;

FIG. 8 shows the ejecting mechanism of the device of FIG. 6;

FIG. 9 shows a surgical filament in accordance with one embodiment of the invention;

FIG. 10 shows a surgical filament in accordance with another embodiment of the invention;

FIG. 11 shows fastening a surgical filament at two locations in a body cavity;

FIG. 12 shows a surgical fastening device according to a second embodiment of the invention;

FIG. 13 shows the barrel of the device of FIG. 12;

FIG. 14 shows the ejection mechanism of the device of FIG. 12;

FIG. 15 shows the cutter of the device of FIG. 12;

FIG. 16 shows a surgical fastener according to a sixth embodiment of the invention;

FIG. 17 shows a surgical fastening device according to a third embodiment of the invention;

FIG. 18 shows a surgical filament according to a third embodiment of the invention;

FIG. 19 shows the barrel of the device of FIG. 17;

FIG. 20 shows the ejecting mechanism of the device of FIG. 17;

FIG. 21 shows a surgical fastener according to a seventh embodiment of the invention;

FIG. 22 shows a surgical fastening device according to a fourth embodiment of the invention;

FIG. 23 shows the central control rod of the device of FIG. 22;

FIG. 24 shows the distal end of the barrel of the device of FIG. 22;

FIG. 25 shows the inner sleeve of the device of FIG. 22; and

FIG. 26 shows repair of stress incontinence using a surgical fastening device of the invention.

### DETAILED DESCRIPTION OF THE INVENTION

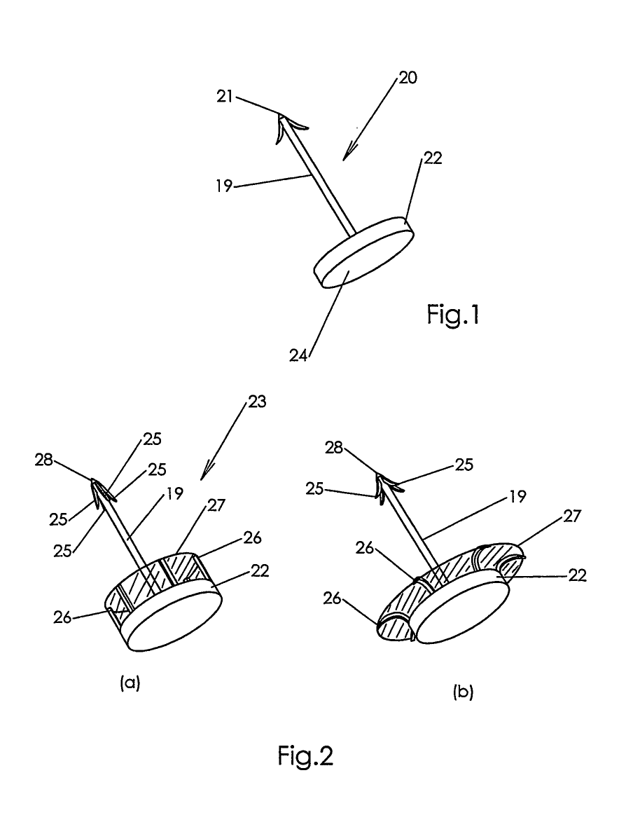

FIG. 1 shows a fastener 20 in accordance with one embodiment of the invention. The fastener 20 is preferably made from a bio-compatible material such as stainless steel or Nitinol™. The fastener 20 has a prong 19 that terminates in a barbed tip 21. The barbed tip 21 serves to anchor the fastener 20 in a body tissue when inserted into the tissue, as described below. The fastener 20 also has a tail portion 22 in the shape of a flat disc from which the prong 19 extends. As explained below, the fastener 20 is inserted into a body tissue by applying a force to a surface 24 of the disc 22 so as to impart a kinetic energy to the fastener 20 and cause the barbed tip 21 to enter the body tissue and become affixed in the tissue. The force applied to the surface 24 may arise, for example, from a compressed fluid or a compressed spring applied to the surface 24.

FIG. 2 shows another embodiment 23 of the fastener of the invention in which a tip portion 28 of a prong 19 is provided with two or more pairs of spring-like barbs 25. The barbs 25 are constrained in a compressed configuration shown in FIG. 2a during insertion of the tip portion 28 into a body tissue. The barbs 25 expand into the expanded configuration shown in FIG. 2b after insertion of the tip portion 28 into a body tissue. The fastener 23 preferably has 1 to 10 pairs of barbs 25, and more preferably 2 to 4 pairs of barbs 25.

The fastener 23 also has a tail portion 22 in the form of a disc to which a force is applied during insertion of the tip portion 28 into a body tissue, as explained above in reference to the embodiment of FIG. 1. The disc 22 is provided with flexible tail members 26. The tail members 26 are constrained in the compressed configuration shown in FIG. 2a prior to insertion of the distal portion 28 into a body tissue, and expand into the expanded configuration shown in FIG. 2b in order to prevent the disc 22 from entering the tissue. The flexible members 26 can be covered with flexible sheet 27.

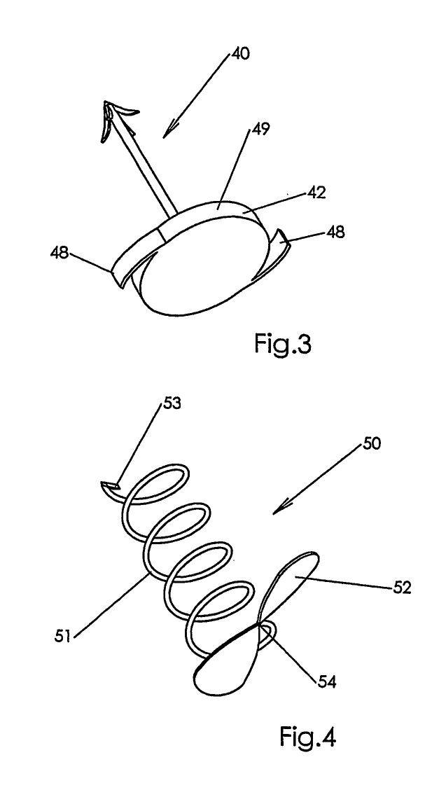

FIG. 3 shows a fastener 40 in accordance with another embodiment of this aspect of the invention. The fastener 40 has a tail end 42 having a disc shape. The disc 42 is provided with two spring-like arcuate fins 48 extending from an edge 49 of the disc 42. The fins 48 are initially constrained against the edge 49 when in a fastening device (not shown), and open into the configuration shown in FIG. 3 when released from the fastening device in order to prevent penetration of the tail end 42 from penetrating the body tissue.

FIG. 4 shows a fastener 50 in accordance with another embodiment of the invention. The fastener 50 comprises a helical coil 51. A tip end 52 of the helical coil 51 is provided with a barb 53 for penetrating a body tissue and becoming anchored in the tissue. A tail end 54 of the helical coil is provided with a propeller 52. When the fastener 50 is propelled towards a body tissue by applying a force to the tail end 54, the propeller 54 causes the fastener 50 to rotate so as to allow the tip end to screw into the body tissue. The helical coil 51 may compress as it enters a tissue.

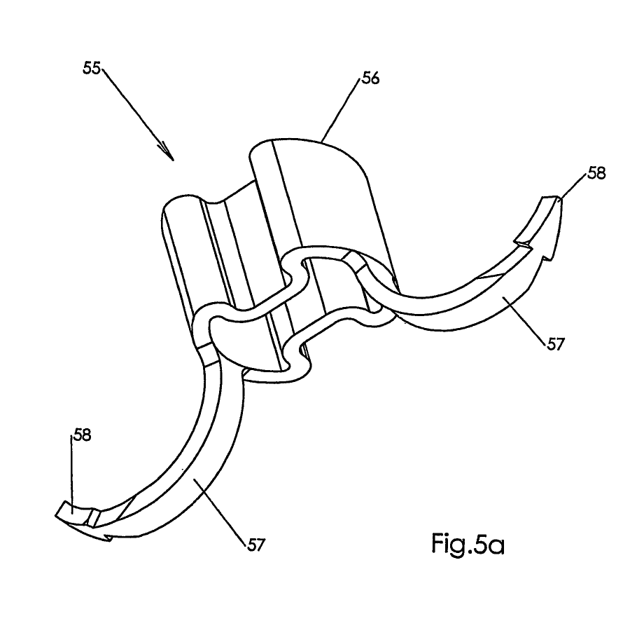

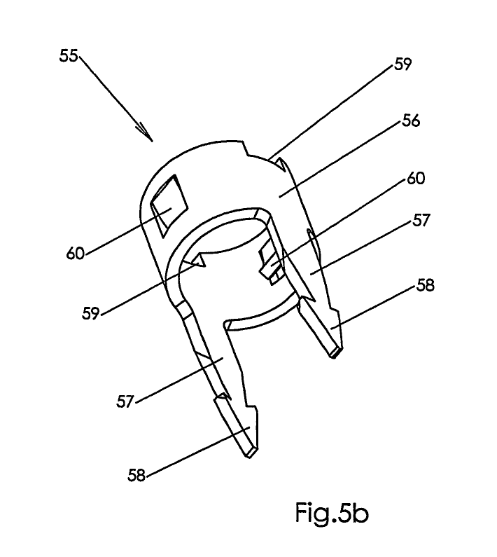

FIG. 5 shows a fastener 55 in accordance with another embodiment of the invention. The fastener 55 has a ring portion 56 from which extend two prongs 57. The prongs 57 terminate in a barb 58. The fastener 55 is formed from a biocompatible elastic or spring-like material such as stainless steel. The fastener 55 has a resting, or unconstrained configuration shown in FIG. 5a, in which the prongs 57 curve outwards from the ring portion 56. As explained below, for insertion into a body tissue, the fastener 55 is constrained in a configuration shown in FIG. 5b in which the prongs 57 are straight. As the prongs 57 enter the body tissue, the prongs 57 revert to the unconstrained configuration shown in FIG. 5a. The ring portion 56 may originally have a circular cross section, as shown in FIG. 5b, and may be deformed into an “I” shaped cross section shown in FIG. 5a in order to grasp a surgical filament, as described below. Alternatively, as shown in FIG. 5b, two diametrically opposed projections 60 may be cut in the ring portion 56. The projections are constrained not to extend from the ring portion 56 when loaded in a fastening device, and spontaneously project inward, as shown in FIG. 5c when released from the fastening device in order to grasp a surgical filament.

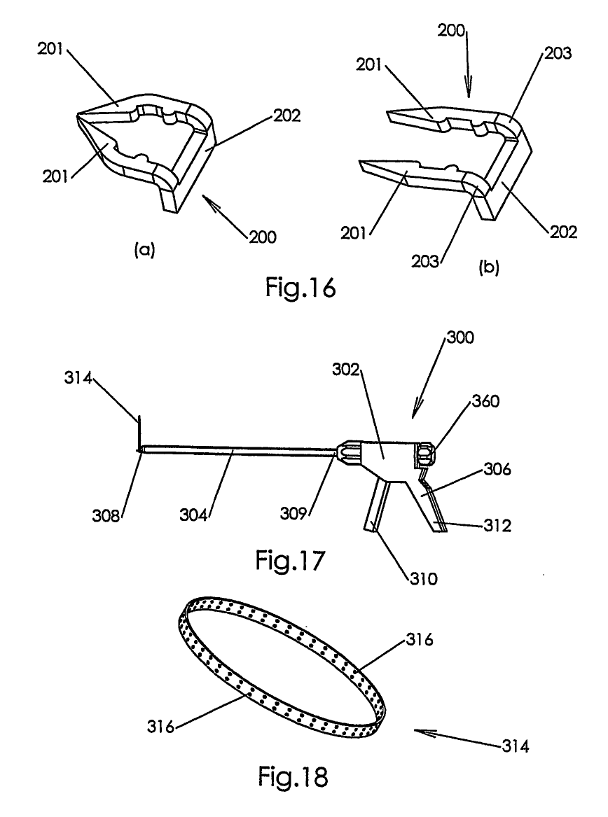

FIG. 16 shows a fastener 200 in accordance with another embodiment of the invention. The fastener 200 has two barbed prongs 201 extending from a crown 202. The fastener 200 has an unconstrained configuration shown in FIG. 16a in which the prongs 201 are directed towards each other. When the fastener is loaded into a fastening device, it is brought into a constrained state shown in FIG. 16b. When the fastener is subsequently ejected from the fastener device, it spontaneously assumes the unconstrained configuration shown in FIG. 16a in order to be anchored in a body tissue. In the constrained state shown in FIG. 16b, the prongs 201 are attached to the crown 202 at a curved regions 203, so the prongs 201 and the crowns 202 do not lie in the same plane.

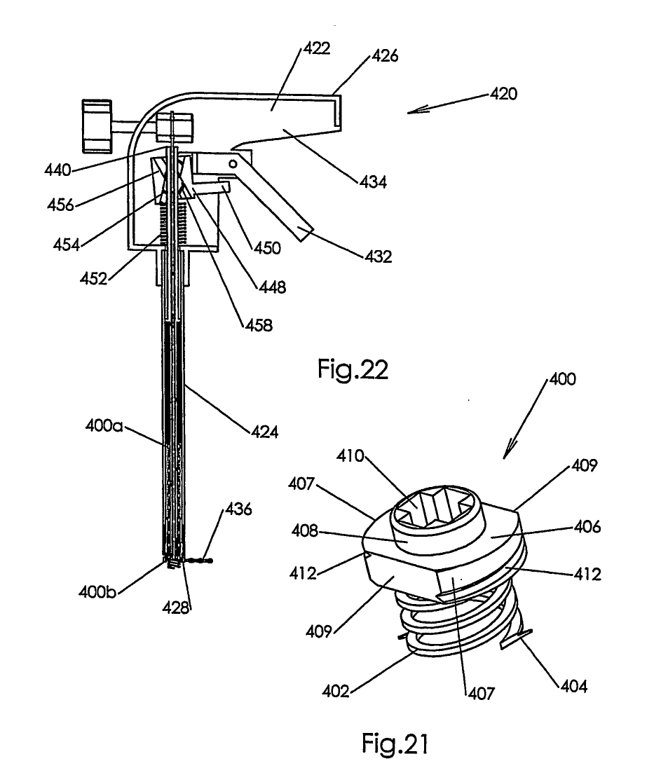

FIG. 21 shows a fastener 400 in accordance with another embodiment of the invention. The fastener 400 comprises a helical portion 402 that terminates in a barbed end 404. The helical portion 402 extends from, and is firmly attached to a D-type fitting 406. The edge of the fitting 406 thus consists of two cylindrical surfaces 407 and two planar surfaces 409. A cap 408 mounted on the fitting 406 has a cross-shaped socket 410. The socket 410 is configured to receive a complementary cross-shaped driving rod in order to rotate the fastener 402 so as to cause the fastener 400 to be screwed into a body tissue. The fitting 406 has two external screw threads 412, that mates with internal screw threads in a fastening device as explained below.

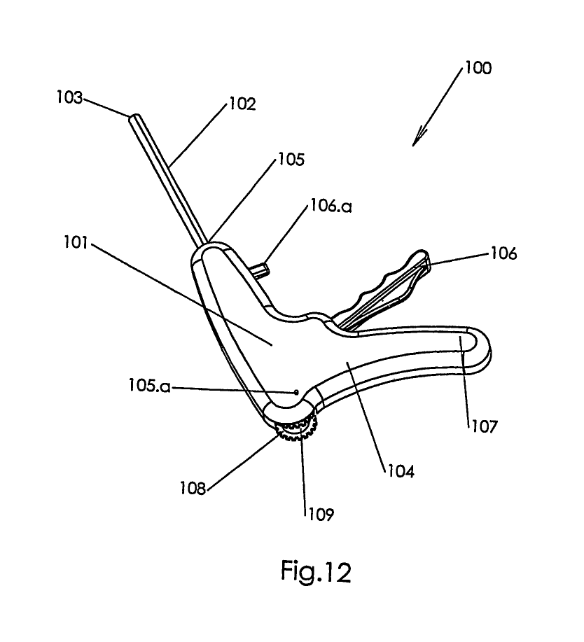

FIG. 12 shows a surgical fastening device 100 in accordance with one embodiment of the invention. The fastening device 100 is used to insert the fastener 55 (FIG. 5) into a body tissue. The fastening device 100 includes a cylindrical barrel 102 in which a plurality of fasteners 55 are stored, as described in detail below. The barrel 102 has a distal end 103 and a proximal end 105.

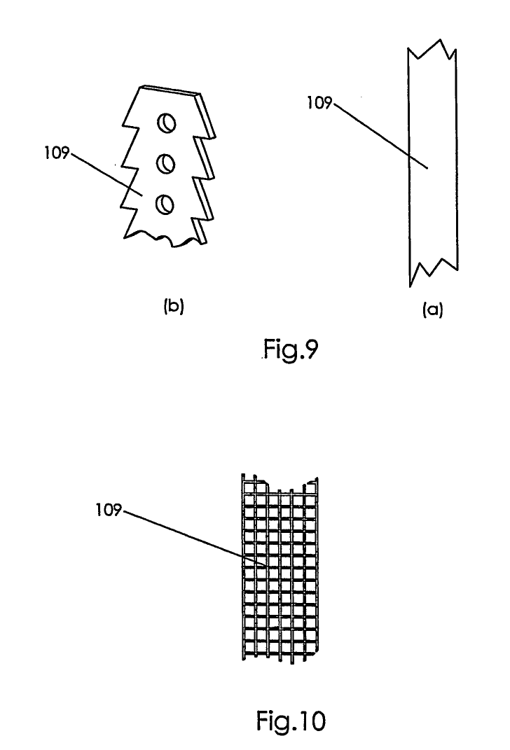

The fastening device 100 has a grasping handle 101 from which the barrel 102 extends. The grasping handle 101 has a housing 104 enclosing an ejecting mechanism for ejecting a fastener 55 in the barrel 102 into a body tissue, as explained in detail below. The ejecting mechanism is activated by a user squeezing a trigger 106 towards a handle portion 107 of the housing 104. The housing 104 also encloses a reel 108 of a surgical filament 109 that is used for joining body tissues together. A terminal segment of the filament 109 extends from the reel 108, passing through the housing and barrel to the distal end of the barrel 102. A portion of the reel 108 extends out of the housing 104 in order to allow a user to manually roll the reel so as to rewind filament back onto the reel. A locking pin 105a allows a user to lock the reel 108 so as to prevent rotation of the reel 108. The filament 109 may be a solid band, as shown in FIG. 9a. Alternatively, the filament may have holes along its length as shown in FIG. 9b. The filament 109 may also be a mesh, as shown in FIG. 10.

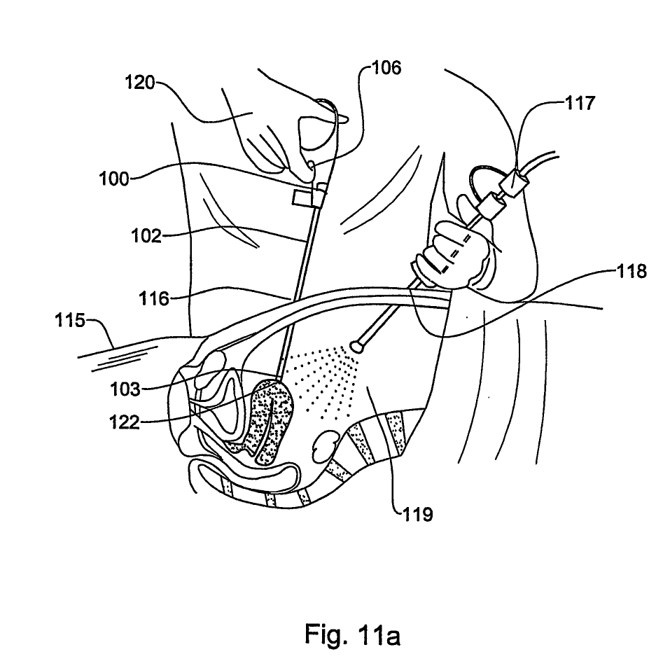

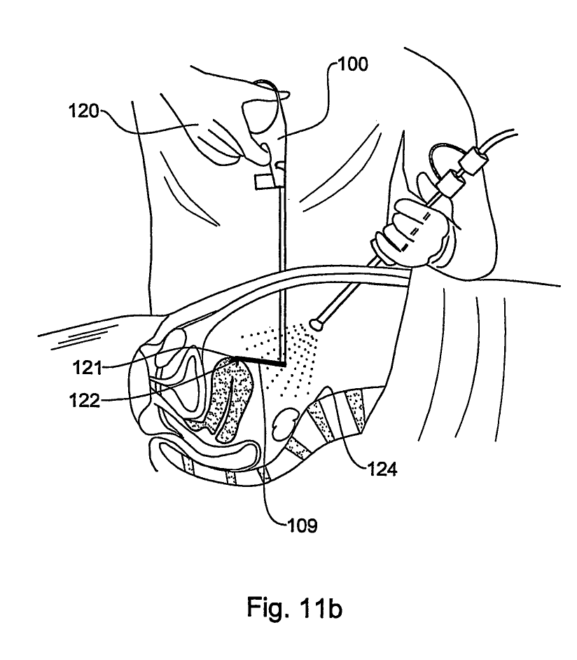

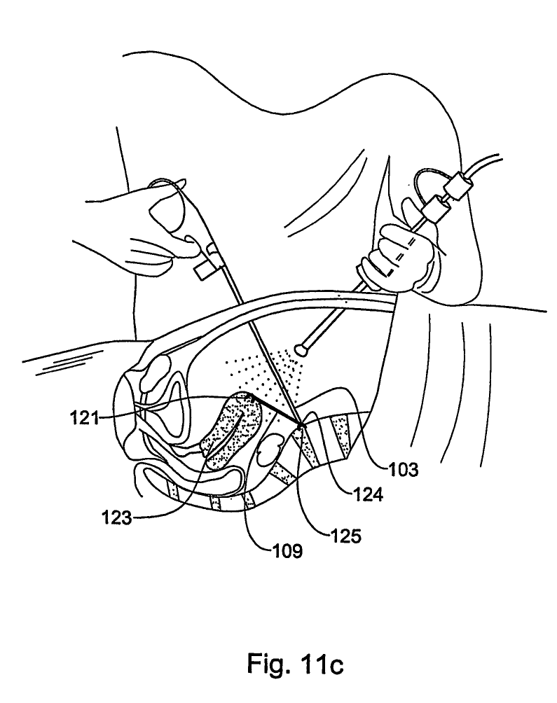

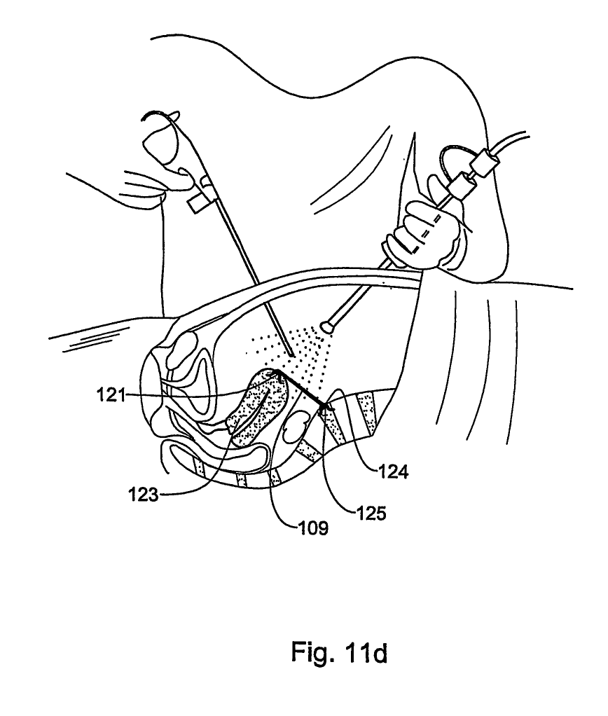

FIG. 11 depicts a surgical procedure in which a surgical fastening device, such as the fastening device 100, is used to insert a fastener 55 at each of two tissue sites in side a body cavity. As shown in FIG. 11a, the shaft 102 of the device 100 is introduced into a body cavity 119 of a subject 115 through an incision at a first location 116 on the body surface. An endoscope 117 is introduced into the body cavity 119 through a second incision at a second location 118 on the body surface. The endoscope 117 illuminates the body chamber 119 containing the body tissue or tissues into which the fasteners 55 are to be inserted. The endoscope is part of an imaging system that displays on a display screen (not shown), an image of the cavity 119, so as to allow a user 120 to observe the cavity 119 as the fasteners are inserted. The body cavity 119 may temporarily be expanded in order to enhance the maneuverability of the fastening device 100 and the endoscope 117 in the cavity 119.

In FIG. 11a, the distal end 103 of the fastening device 100 has been brought to a first location 122 in the body cavity 119 where a first fastener is to be inserted. The user then squeezes the trigger 106 against the handle 107 so as to activate the fastener ejecting mechanism, and eject a fastener from the distal end of the shaft into the tissue at the first location 122. As the fastener is ejected from the distal end of the shaft, the fastener firmly grasps the free end of the filament 109 at a location on the filament 109 adjacent to the distal end 103, so that the free end of the filament becomes pinned to the tissue at the first location 122.

FIG. 11b shows the filament after its free end has been pinned to body tissue at the first location 122 by a fastener 121. As the user then moves the distal end of the shaft away from the first location 122, filament 109 is fed from the reel 108 located in the grasping handle and through the shaft 102. As the distal end 103 approaches a second location 124, the reel 108 may be locked in order to prevent additional strip 109 from being released from the reel 108 by depressing the locking pin 105a on the housing 104 (FIG. 12). As the distal end 103 is further moved towards the second location 124, the first location 121 is pulled towards the second location 122 so as to displace a body organ, such as the body organ 123 towards the second location 124. Before ejecting a fastener at the second location 122, the user may manually rotate the reel 108 in order to retract an amount of the filament back onto the reel so that the filament is stretched taut between the first and second locations.

FIG. 11c shows the fastening device 100 after the distal end 103 has been brought to the second location 124 in the body cavity 119 where a second fastener is to be inserted into body tissue. As can be seen in FIG. 11c, the filament at this time extends from the first location 122 (where the strip is pinned to body tissue by the fastener 121) to the second location 124. The user then activates the fastener ejecting mechanism again to eject a second fastener at the second location. As the second fastener is ejected from the shaft, it grasps the filament in the vicinity of the distal end of the shaft, so as to pin the strip, at the second location.

The fastening device 100 includes a cutter, to be described below, located at the distal end of the shaft for cutting the filament 109. FIG. 1 id shows the strip 109 after having been cut by the cutter so as to release a segment of filament 109 pinned at its ends at the first and second locations 122 and 124 by the fasteners 121 and 125, respectively. This process may be repeated as required during a single surgical procedure so as to deploy any number of filament pieces in the body cavity, so as to fix a body organ, such as the organ 123 in a desired position in the body cavity.

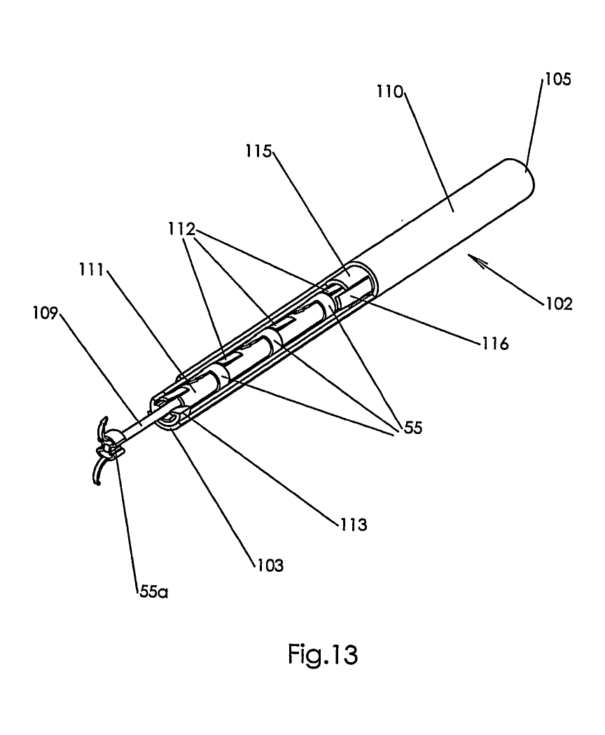

FIG. 13 shows an interior view of the shaft 102 of the fastening device 100. The shaft 102 comprises three coaxial sleeves. A middle sleeve 115 is located between an outer sleeve 110 and an inner sleeve 111. The inner sleeve 111 is separated from the middle sleeve 115 by an annular gap. A plurality of fasteners 55 in the constrained configuration shown in FIG. 5b are mounted on the inner sleeve 110 with the inner sleeve 110 passing through the ring portion 56 of the fasteners 55 (FIG. 5), so that the fasteners 55 are located in the annular gap between the inner and middle sleeves 111 and 115, respectively. The inner sleeve is provided with a sequence of ratchet projections 112 that push the fasteners 55 in a distal direction when the inner sleeve 110 is pushed in the distal direction by an ejecting mechanism described below. The fasteners 55 may be provided with notches 59 in the ring region 56 (FIG. 5) to receive the tips of the projections 112 in order to prevent rotation of the fasteners 55 in the barrel during longitudinal displacement of the fasteners in the barrel. The middle sleeve 115 is also provided with spring-like projections 116 that prevent longitudinal movement of the fasteners 55 towards the proximal end 105 of the barrel when the inner sleeve 110 moves longitudinally towards the proximal end 105 of the barrel after a fastener has been ejected, as explained below.

The ejecting mechanism is configured to apply a force to the proximal end of the inner sleeve 110 so as to cause the entire stack to move one ratchet unit towards the distal end 103 of the shaft 102. As shown in FIG. 13, the distal most fastener 55a in the stack is thus ejected from the distal end of the shaft 103. At the distal end 103 of the shaft 102 is a fastener deformer 113. As the distal most fastener 55 is ejected from the distal end of the shaft 102, the fastener passes through the fastener deformer 113 causing the ring portion 56 to be deformed from the circular shape shown in FIG. 5b to the “I” shape shown in FIG. 5a. Deforming the circular portion 56 to the “I” shape causes the circular portion 56 to pinch and firmly grasp the free end of the filament 109. The ejected fastener thus pulls the free end of the filament 109 as it is ejected, as shown in FIG. 13. As the end of the filament 109 is pulled by the ejected fastener, the spool 108 rotates so as to release more filament. Alternatively, the projections 59 (FIG. 5) may be used to grasp the filament 109.

After the fastener 55 has been ejected from the shaft 102, the prongs 57 spontaneously revert to their unconstrained configuration shown in FIG. 5a, so as to allow the fastener to become further attached at a specific location on a body tissue (the body tissue is not shown in FIG. 13). The free end of the filament 109 is thus also firmly attached to the same location on the tissue.

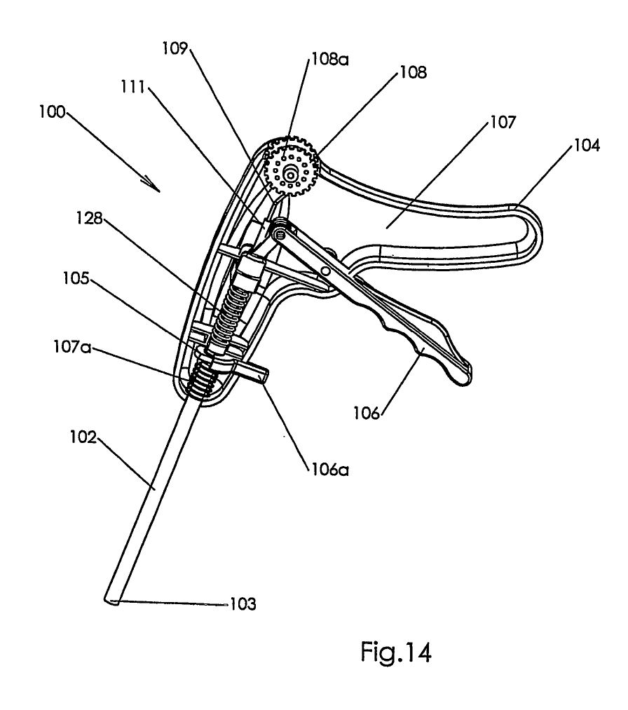

FIG. 14 shows an interior view of the grasping handle 107. The interior sleeve 111 extends from the shaft 102 into the grasping handle 107 and terminates near the reel 108. The filament 109 extends from the reel 108 and is conducted through the interior sleeve 111 to the distal end 103 of the shaft 102. The trigger 106 is spring biased in a released position shown in FIG. 14 by means of a restoring spring 128. Squeezing the trigger 107 applies a force to the proximal end of the inner sleeve 110 in order to eject a fastener, as explained above. The reel 108 is provided with locking holes 108a configured to receive the locking pin 105 (FIG. 12).

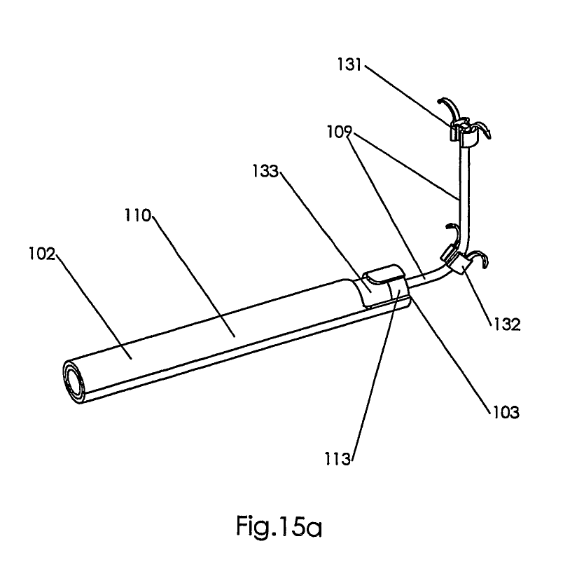

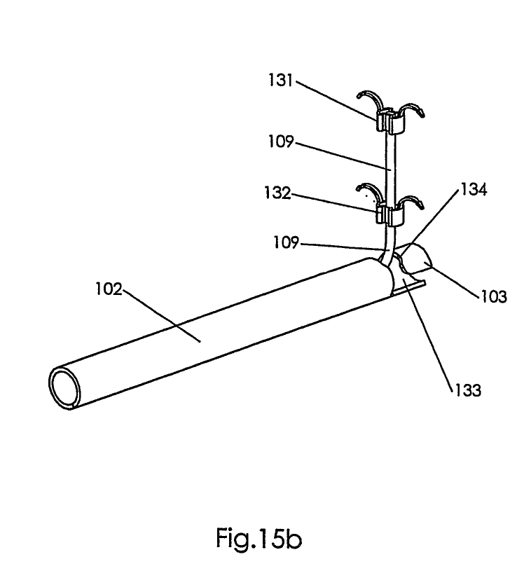

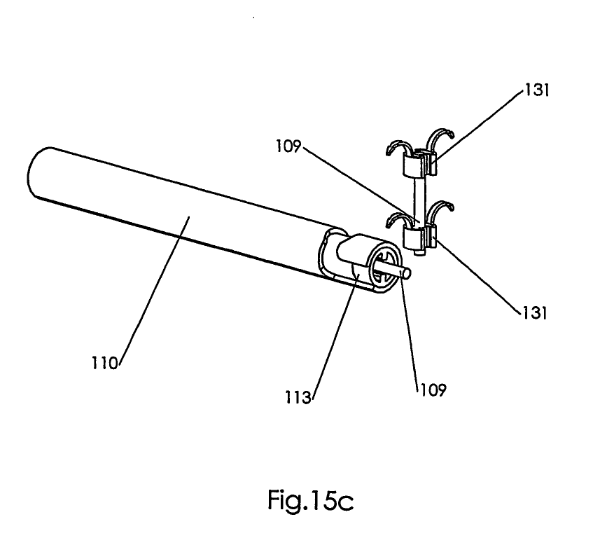

FIG. 15 shows the cutter located at the distal end 103 of the shaft 102 for cutting the filament 109. FIG. 15a shows a segment of the filament 109 after being pinned to body tissue (body tissue not shown in FIG. 15) at two locations by a pair of fasteners 131 and 132. The cutter consists of an “L” shaped notch 133 formed in the outer sleeve 110 extending from the distal end 103 of the shaft 102. A lever 106a (FIGS. 12 and 14) is rotated about the barrel 102 in order to displace the outer sleeve 110 longitudinally towards the distal end of the barrel so that it extends beyond the deformer 113 as shown in FIG. 15b. The user then manipulates the distal end 103 so as to bring the filament 109 into the notch 133, as shown in FIG. 15b. The lever 106a is then released. Under the influence of a restoring spring 107a, the lever 106.5 returns to its original position causing the outer sleeve 110 to move longitudinally towards the proximal end of the barrel. The filament 109 thus becomes sheared between a cutting edge 134 on the notch 133 and the deformer 113, as shown in FIG. 15c.

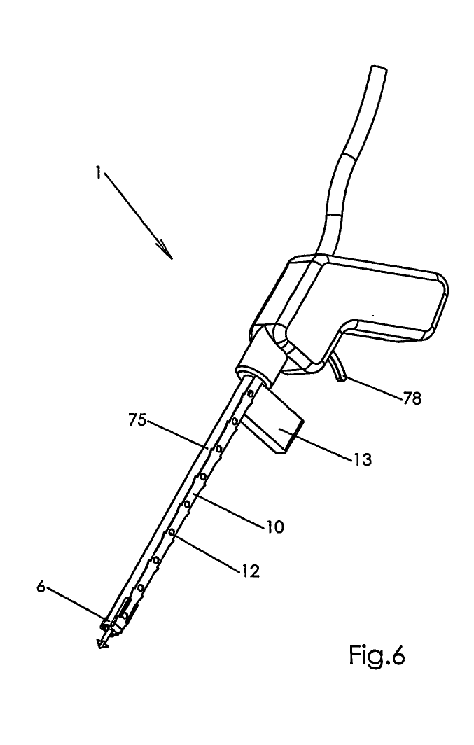

FIGS. 6, 7, and 8 show a fastening device 1 for inserting one or more fasteners in to a body tissue in accordance with another embodiment of the invention. The fastening device 1 may be used, for example, to insert any one of the fasteners 20, 28, 40, or 52. The fastening device 1 includes an insertion opening 62 configured to receive a magazine 63 containing plurality of fasteners 64. Each fastener 64 is encased in a metal shell 69. The shells 69 all have the same dimensions that are determined by the inner dimensions of the magazine 63 and a barrel 75. A shell 69 can, however, accommodate fasteners of different sizes and shapes. In this way, the magazine 63 can be loaded with fasteners of different shapes, as required in any application.

The magazine 63 can preferably hold 5 to 40 shells 69, and more preferably 10 to 20 shells 69, each shell containing a fastener 65. The shells 69 are fed one at a time, by a metal spring in the magazine 63 towards the barrel 75. After ejecting a fastener 65, the shell of the ejected fastener is ejected out of the barrel 75, and may be collected separately in a bag or can.

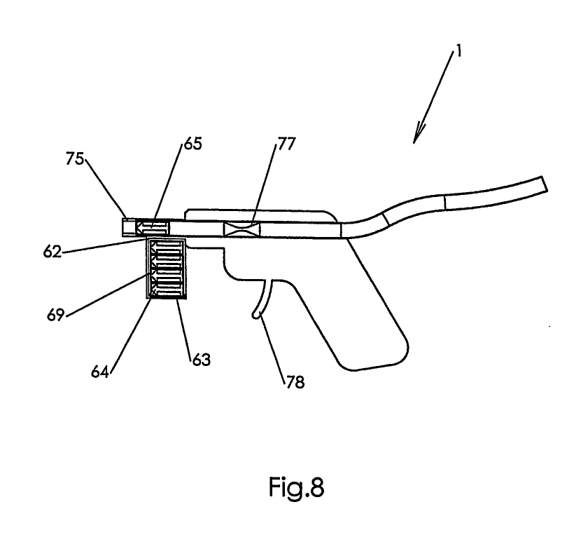

The fastening device 1 also includes an ejecting mechanism to impart kinetic energy to a fastener so as to eject a fastener from the fastening device through the barrel 75. The ejecting mechanism may be mechanical (i.e. by means of a spring). Alternatively, as shown in FIG. 8, the ejecting mechanism may be a pneumatic or hydraulic mechanism. Depression of a trigger 78 releases a pneumatic valve 77 causing a compressed gas, such as compressed air, or a pressurized liquid fluid to be delivered from a source (not shown) into the barrel 75 of the fastening device 1 which imparts a kinetic energy to a fastener 65 in the barrel 75. In this way, the fastener 65 is ejected from the fastening device 1.

The barrel 75 of the fastening device 1 can be of a smaller diameter than the barrel diameter of conventional fastening instruments, due to the absence of a mechanical feeding mechanism or fastener deforming mechanism in the barrel 75. For example, if the maximum external diameter of a fastener is 1 mm, then the external diameter of the barrel 75 can be as small as 2 mm.

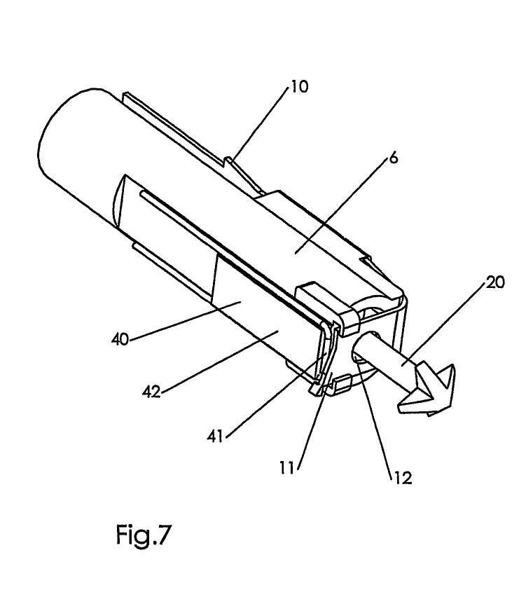

The fastening device 1 also includes a cartridge 13 containing a reel of surgical filament 10. If the filament 10 is attached to the barrel 75, a cover tube (not shown) of up to 10 mm, and preferably up to 5 mm, outside diameter can contain both the barrel 75 and the filament 10. The filament is pulled out along the barrel 75 before using the fastening device 1, until the end 11 covers the distal end 6 of the barrel 5 as shown in FIG. 7. The filament 10 is provided with a series of holes. The filament passes through the trajectory of a fastener 20 as the fastener passes through the barrel so that the tip of the fastener passes through a hole 12 in the filament. The tail 22 of the fastener cannot pass through the hole so that the end of the filament becomes pinned to the tissue as the fastener penetrates the tissue. An amount of filament may then be released from the reel and a second fastener ejected. The filament thus becomes pinned at two locations in body tissue.

The filament is then cut by a cutting mechanism 40. The fastening device 1 includes a cutting mechanism 40 to cut the filament 10 after affixing the filament at two locations to body tissue. As shown in FIG. 7, the cutting mechanism can be by means of mechanical scissors, a hot wire system or a hot RF tip element 41 that is isolated by a plate 42 from the barrel 5. The electrical energy for the cutting element can be from an external source such as an electrical power supply or from an RF generator, or can be an internal device battery. After cutting the detached piece of filament connects two tissue locations so as to provide support for a tissue or to hold two tissues together.

FIG. 17 shows a surgical fastening device 300 in accordance with another embodiment of the invention. The fastening device 300 is used to insert the fastener 200 (FIG. 16) into a body tissue. The fastening device 300 has a grasping handle 302 and a barrel 304. The barrel 304 has a distal end 308 and a proximal end 309. The grasping handle 302 has a housing 306 containing an ejecting mechanism for ejecting fasteners from the distal end 308 of the barrel 304. The ejecting mechanism is activated by squeezing a trigger 310 towards a handle portion 312 of the grasping handle, as explained below. A fastener ejected from the distal end 308 pins a filament 314 to a body tissue

FIG. 18 shows the filament 314. Initially, the filament 314 is a closed loop. The filament 314 has two rows of perforations 316 along its length. The filament 314 is loaded onto the fastening device 300 so that the filament 314 is loops around the proximal end 309 and the distal end 308 of the barrel 304.

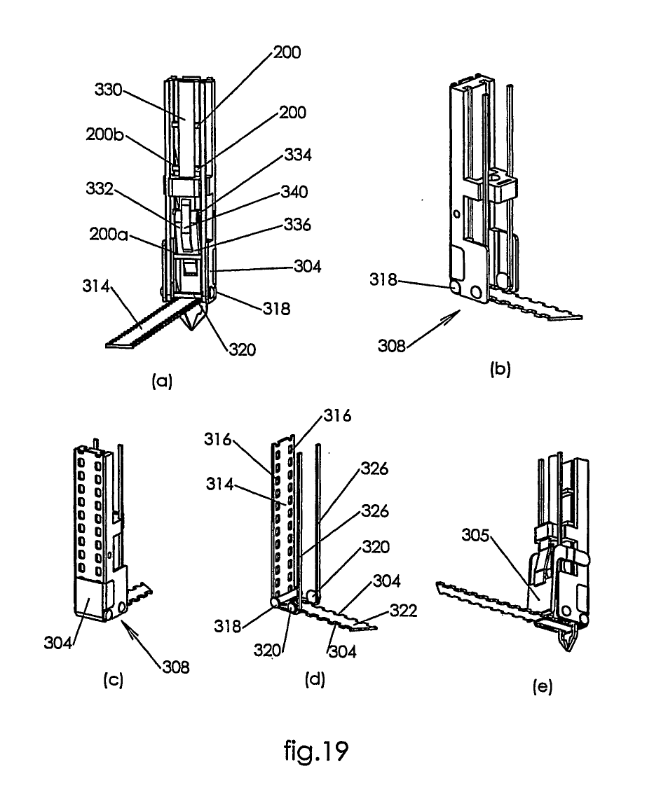

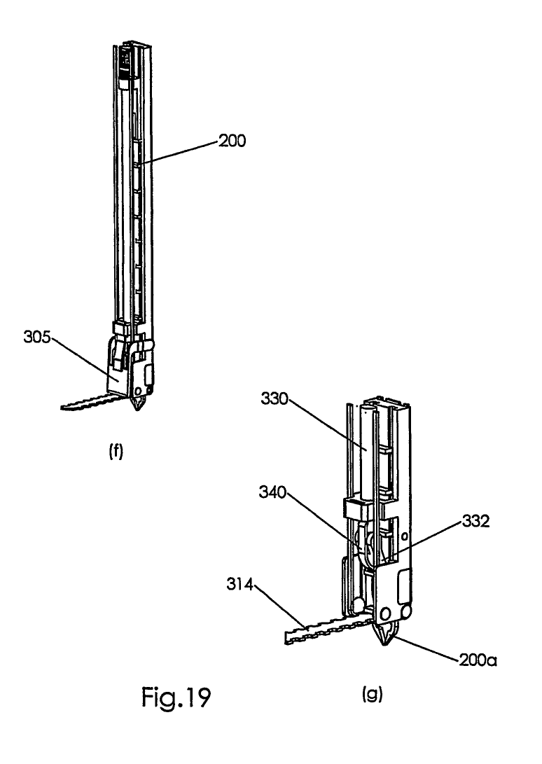

FIGS. 19a to 19g show the distal end 308 of the barrel 304 in greater detail from several perspectives. The barrel 304 contains a stack of fasteners 200. The fasteners 200 in the barrel 304 are in the constrained state shown in FIG. 16b.

At the distal end 308 of the barrel 304 is a first roller 318 and a pair of roller cutters 320. As the filament 314 moves in the shaft 304, the roller cutters 320 cut the filament 314 along the perforation 316. A portion 322 of the filament 314 is thus released from distal end 308 having a series of notches 304 along each edge. Residual fibers 326 continue to the proximal end 304 of the shaft.

A central rod 330 extends through the length of the barrel 304. A tongue 332 is hinged to the distal end of the rod 330 at an axis 334. The distal edge 336 of the tongue 332 contacts the crown 202 (FIG. 16) of the distal most fastener 200a. A flat spring 340 extends from the distal end of the rod 330 and maintains the distal edge 336 of the tongue 332 in contact with the crown 202. The distal end of the barrel 304 is provided with a cutter 305 for cutting the filament 314. The cutter may be, for example, a blade, an radiofrequency (RF) cutter, or a hot wire cutter.

Squeezing of the trigger 310 (FIG. 17) causes displacement of the rod 330 towards the distal end 308 of the barrel 304. This movement of the rod 330, in turn, causes the distal most fastener 200a to be ejected from the distal end 308. As the distal most fastener is ejected from the distal end, prongs 201 pass through notches 304 on the edges of the filament 314, and then reverts to its unconstrained configuration (FIG. 16a), so as to be fixed in a body tissue. (The body tissue is not shown in FIG. 19).

Releasing the trigger 310 then causes the rod 330 to be displaced longitudinally towards the proximal end 309 of the barrel 304. The stack of fasteners 200 is then displaced in the barrel 304 towards the distal end 308. As the fastener 200b slides by the tongue 332, the tongue rotates slightly around the axis 334 against the flat spring 340. When the crown 200 of the fastener 200b has passed the distal edge 336 of the tongue 332, the distal edge 336 rotates about the axis 334 under the influence of the spring 340, so that the distal edge 336 is directly above, and in contact with the crown 200 of the fastener 200b. Squeezing the trigger 310 would then cause the fastener 200b to be ejected from the distal end 308, as explained above.

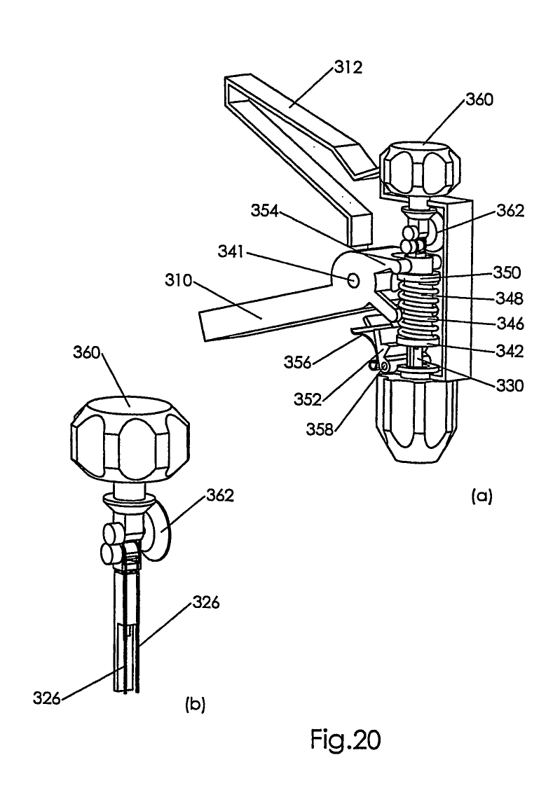

FIGS. 20a and 20b show the grasping handle 302 of the fastening device 300, with the housing 306 removed for clarity. The central rod 330 extends into the grasping handle 302 at its proximal end. An annular ledge 342, attached to the rod 330 supports a helical spring 346 surrounding a widened portion 348 of the rod 330. Squeezing the trigger 310 against the handle portion 312 causes a first extension 354 of the trigger 310 to depress an annular ring 350 surrounding the rod 330 and mounted on the helical spring 346. A stop 352 under the ledge 342 prevents the rod from being displaced longitudinally in the distal direction as the trigger is being squeezed. This causes the helical spring 346 to be compressed. As the trigger 310 continues to be squeezed, a second extension 356 of the trigger 310 contacts the stop 352 causing the stop 352 to rotate about an axis 358 out from underneath the ledge 342 so as to allow the compressed spring 348 to rapidly revert to its resting state, so as to drive the rod 330 distally to eject a fastener as described above.

The grasping handle 302 also includes a knob 360 (FIGS. 17, 20a and 20b) for maintaining a desired tension in the filament 314. A knob 362 is used to lock the filament 314

FIG. 22 shows a fastening device 420 in accordance with another embodiment of the invention. The fastening device 420 may be used to insert the fastener 400 into a body tissue. The fastening device 420 has a grasping handle 422 from which extends a barrel 424. The barrel 424 has a distal end 428 and a proximal end 430 located inside the grasping handle. The grasping handle 422 has a housing 426 enclosing an ejecting mechanism for ejecting a fastener 400 from the distal end 428 of the barrel 424. The ejecting mechanism is activated by squeezing a trigger 432 against a handle portion 434 of the grasping handle 422. A fastener 400 ejected from the distal end 428 pins a strip 436 to a body tissue, as explained below.

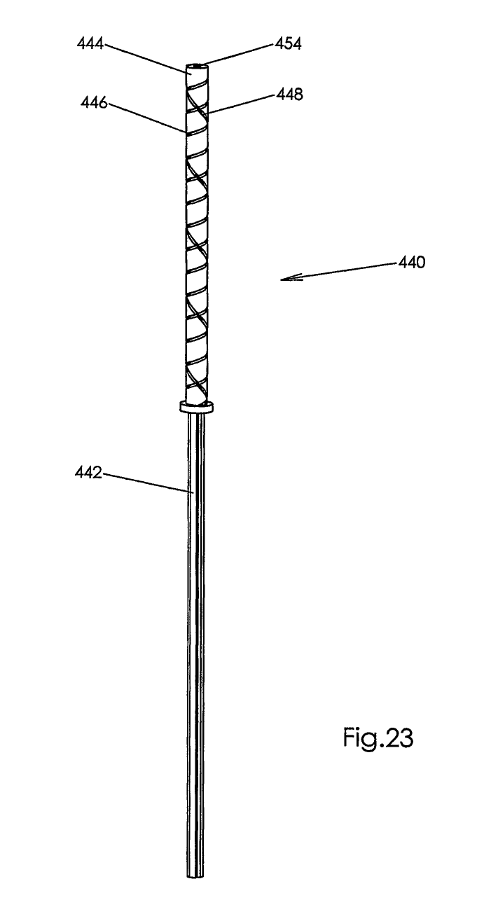

A central rod 440 shown in greater detail in FIG. 23 extends from the grasping handle 422 into the barrel and terminates near the uppermost fastener 400a in the barrel 424 to the distal end 428. The rod 440 has a distal portion 442 and a proximal portion 444. The distal portion 442 has a cross-shaped cross section and is configured to be received in the cross-shaped socket 410 of the fastener 400 (FIG. 21). The distal portion is located inside the barrel 424. The proximal portion 444 is located in the grasping handle 422 and has a circular cross-section. The proximal portion 444 is provided with a helical screw thread 446. The proximal portion is also provided with a helical groove 448 having a pitch that is greater than the pitch of the helical screw thread 446. A hole 454 extends through the rod 440.

The grasping handle 422 includes a selector 448. The selector 448 has an integral lever 450 extending out of the housing 426. The selector 448 can alternate between a first position shown in FIG. 22 in which the lever 450 is raised and a second position (not shown) in which the lever 450 is lowered. The lever 450 is spring biased in the lowered position by means of a helical spring 452 surrounding the rod 440.

The selector 448 has a control cavity 456 surrounding the rod 440. Raising the lever 450 against the spring 452 brings the selector into the configuration shown in FIG. 22, in which inner screw threads 454 on a portion of the central cavity 445 engage the outer helical threads 446 on the proximal portion 444 of the rod 440 (FIG. 23). Squeezing the trigger 432 with the lever 450 held in its raised position causes the rod 440 to be displaced longitudinally towards the distal end 428. The distal end of the rod is then received in the socket 410 of the proximal most fastener 400a in the barrel 424. The lever 450 is then released, so as to return to its lowered position under the influence of the spring 452. In this configuration, the selector 448 is in its second configuration in which a second inner screw thread 458 engages the helical groove 448 on the proximal portion of the rod 440. The trigger 432 is then released causing the rod 440 to rotate. Since the distal end of the rod 440 is inserted in the socket 410 of the uppermost fastener 400a in the barrel, rotation of the rod 410 drives the rotation of the uppermost fastener 400a.

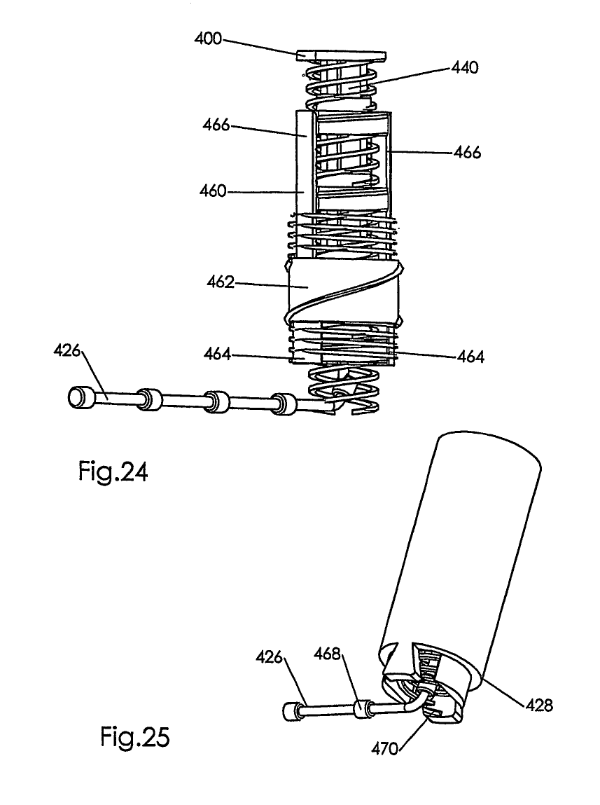

As shown in FIG. 24, an inner sleeve 460 is located inside the barrel 424. The inner sleeve surrounds the stack of fasteners 400. The inner sleeve 460 has a cylindrical portion 462. Two diametrically opposed cutting blades 464 extend from below the cylindrical portion 462 for cutting the filament 426. Two diametrically opposed projections 466 extend above the cylindrical portion 462. The projections 466 are planar and are parallel to each other and extend along the entire length of the barrel 424. The stack of fasteners 400 is oriented between the projectors 466 with the planar surfaces 409 of the fasteners 400 parallel to the planar projectors 466 of the inner sleeve 460. Thus, rotating of the uppermost fastener 400a in the stack by the rod 440, as explained above, causes the inner sleeve 460 to rotate, which in turn, causes all of the fasteners 400 in the stack to rotate. Rotation of the lowermost fastener 400b in the stack causes the helical portion 402 of the lowermost fastener 400b to screw into a body tissue.

FIG. 25 shows the distal end 428 of the barrel in greater detail. An inner screw thread 470 on the inner surface of the barrel 424 engages the outer screw thread 412 on the fitting 407 of the lowermost fastener 400 (FIG. 21) in order to prevent the lower most fastener 400b from inadvertently falling out from the distal end 428 of the barrel. The lowermost fastener 400b can thus only be ejected from the distal end when rotated by the rod 440, as explained above.

The filament 426 passes through the stack of fasteners 400 and extends beyond the distal end 428. The filament has a circular cross-section and has bulges 468 periodically placed along its length. For example, a bulge 468 may be located every centimeter along the length of the filament 426.

The filament 426 has a diameter that is less than the spacing of the turns of the helical portion 402 of the lowermost fastener 400b. The bulges 468, however, are too wide to pass between the turns of the helical portion 402 of the lowermost fasteners 400b. Thus, as the lowermost fastener 400b is ejected from the distal end 428 of the barrel, and screws into a body tissue, the filament enters in between the turns of the helical position 402 and thus becomes pinned to the body tissue. The presence of the bulges 468 then prevents the filament from slipping under the fastener 400b. When the lever 450 and trigger 432 are then raised, the inner sleeve 460 extends beyond the distal end 428. The cutting blades 464 then cut the filament as shown in FIG. 24.

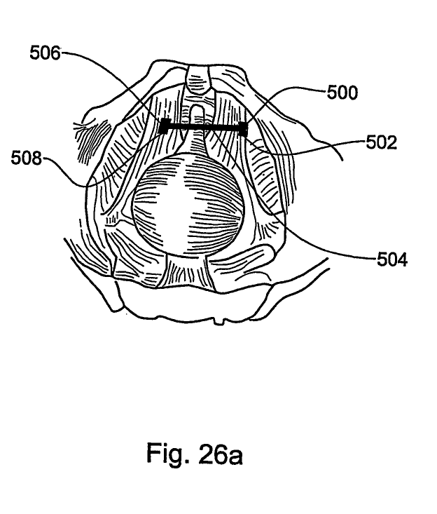

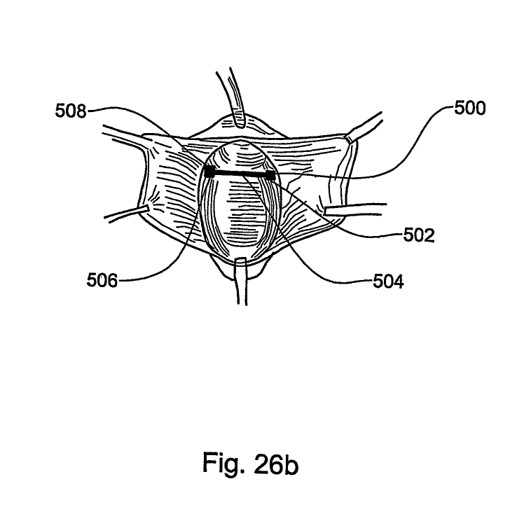

FIG. 26 shows use of the fastening device of the invention in a method of vaginal repair of stress incontinence. The procedure is shown in an abodiminal view in FIG. 26a, and in a vaginal view in FIG. 26b. An incision 5 to 10 mm is made on the anterior vaginal wall over the urethra. A plane is then developed bilaterally between the vaginal wall and the urethopelvic ligament toward the attachment of this ligament to the arcuate ligament of the endopelvic fascia. The fastening device of the invention is introduced through the incision towards the endopelvic fascia. A fastener 502 of the invention is then ejected from the fastening device so as to pin an end of a filament 504 at a first location 500 on the endopelvic fascia on one side. The fastener is then removed through the vaginal incision, and is then reintroduced through the vaginal incision to the opposite endopelvic fascia and a second fastener 506 is ejected from the fastening device so as to pin the filament at a second location 508 on the second side of the endopelvic fascia. The filament 504 is then cut by the fastener, so as to leave a piece of filament stretched between the two endopelvic fascia. The fastener is then removed through the vaginal incision.

1. A surgical fastening device for pinning a surgical filament to a body tissue, comprising:
(a) a grasping handle;
(b) a slender shaft extending from the grasping handle, the shaft having a distal end:
(c) a compartment configured to contain one or more surgical fasteners;
(d) an activatable ejecting mechanism ejecting a surgical fastener from the distal end of the shaft; and
(e) a filament dispensing system configured to dispense surgical filament at the distal end of the shaft, a fastener grasping the filament ejected from the distal end of the shaft.

2. The surgical fastening device according to claim 1 wherein the ejecting mechanism is spring mechanism, a hydraulic mechanism or a pneumatic mechanism.

3. The surgical fastening device according to claim 1 further comprising a cutter for cutting the filament.

4. The surgical fastening device according to claim 3 wherein the cutter comprises a blade, a hot wire, or an RF generator.

5. The surgical fastening device according to claim 1 further comprising a surgical filament.

6. The surgical fastening device according to claim 5 wherein the filament is a mesh, a ribbon, a strip, a wire, a net or a thread.

7. The surgical fastening device according to claim 1 wherein the fasteners are contained in the shaft.

8. The surgical fastening device according to claim 1 further comprising one or more surgical fasteners.

9. The surgical fastening device according to claim 8 wherein the fasteners comprises a barbed prong extending from a disc.

10. The surgical fastening device according to claim 9 wherein the fasteners comprise two or more barbs.

11. The surgical fastening device according to claim 9 wherein the fasteners have spring like fins extending from the disc.

12. The surgical fastening device according to claim 9 wherein the fasteners have barbed projections extending from the disc.

13. The surgical fastening device according to claim 9 wherein the fasteners comprise a helical wire having a first barbed end and a second end attached to a propeller.

14. The surgical fastening device according to claim 8 wherein the fasteners comprise a crown from which extend two prongs.

15. The surgical fastening device according to claim 8 wherein the fasteners comprise a socket configured to receive a rotatable driving rod.

16. The surgical fastening device according to claim 7 further comprising one or more surgical fasteners in the shaft.

17. The surgical fastening device according to claim 16 wherein the fastener has a ring portion from which extend two barbed prongs.

18. The surgical fastening device according to claim 16 wherein the fastener has an unconstrained configuration in which the prongs curve outwards from the ring portion and a constrained state in which the prongs are straight and parallel to a longitudinal axis of the ring portion.

19. The surgical fastening device according to claim 18 wherein the fasteners are maintained in the constrained state in the shaft.

20. The surgical fastening device according to claim 1 wherein a fastener is pinched so as to grasp the filament when being ejected from the shaft.

21. The surgical fastening device according to claim 1 wherein a fastener pierces the filament when being ejected from the shaft.

22. The surgical fastening device according to claim 1 wherein a fastener passes through a hole in the filament when being ejected from the shaft.

23. The surgical fastening device according to claim 1 wherein notches are formed along edges of the filament and prongs of a fastener enter the notches when being ejected from the shaft.

24. The surgical fastening device according to claim 1 wherein the filament has spaced apart bulges.

25. The surgical fastening device according to claim 7 further comprising a ratchet mechanism preventing movement of fasteners in the shaft towards the grasping handle.

26. The surgical fastening device according to claim 1 wherein the ejecting mechanism is located in the grasping handle.

27. The surgical fastening device according to claim 1 configured to screw a fastener into a body tissue.

28. A surgical fastener for use in the surgical fastening device according to claim 1.

29. The surgical fastener according to claim 27 formed from a biodegradable material.

30. The surgical according to claim 27 formed from stainless steel or Nitinol™.

31. A surgical filament for use in the surgical fastening device according to claim 1.

32. The surgical filament according to claim 30 made from a biodegradable material.

33. Use of a surgical fastening device according to claim 1 for attaching a surgical filament to a body tissue.

34. The surgical fastening device according to claim 1 for use in attaching a surgical filament to a body tissue.

35. A method for pinning a surgical filament to a first location of body tissue in a body cavity comprising introducing into the body cavity a surgical fastening device according to claim 1, into the cavity and ejecting a first surgical fastener from the shaft so as to pin a surgical filament to the first location.

36. The method according to claim 35 further comprising ejecting a second surgical fastener from the shaft so as to pin the filament to a second location of body tissue in the cavity.

37. The method according to claim 36 wherein the filament is stretched taut between the first and second locations before the second fastener is ejected.

38. The method according to claim 37 for use in the treatment of stress incontinence, inguinal hernia, pelvic organ prolapse, gastroesophageal reflux, laproscopic anastomoses of a tubular organ, and repair of ureteropelvic obstruction.
